(19) United States
(12) Patent Application Publication
(10) Pub. No.: US 2007/0219446 A1
(43) Pub. Date: Sep. 20, 2007

(54) SYSTEM AND APPARATUS FOR IMAGING AND TREATING HOLLOW BODY CAVITIES

(76) Inventor: Niyazi Beyhan, Santa Clara, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/685,268

(22) Filed: Mar. 13, 2007

Related U.S. Application Data
(60) Provisional application No. 60/782,371, filed on Mar. 14, 2006.

(51) Int. Cl. A61B 8/00; A61N 5/00

(52) U.S. Cl. 600/439; 600/437; 600/3

(57) ABSTRACT

Methods and apparatus for image-guided interstitial radiation are provided. In practicing the subject methods, a catheter is placed in the interstitium in close proximity to the site of tumor. The interstitial catheter is designed to receive an ultrasound imaging probe, and a radioactive source for interstitial radiation therapy. The interstitial catheter may be equipped with a balloon member. Further a system facilitating substance communication with the interstitium is provided.

### CROSS-REFERENCES TO RELATED APPLICATIONS

This application claims the benefit of provisional application 60/782,371 (Attorney Docket No. 026249-000100US), filed on Mar. 14, 2006, the full disclosure of which is incorporated herein by reference.

### BACKGROUND OF THE INVENTION

1. Field of the Invention

The field of this invention is medical devices, more specifically, a device for characterizing and optionally treating tissue.

One common method of delivering radiation to the breast tissue, referred to as Accelerated Partial Breast Irradiation (APBI), relies on placing a balloon catheter into the cavity created at the time of lumpectomy. The balloon catheter is inflated with saline and contrast agent to allow the surrounding tissue to conform to the balloon and radiation delivered to tissue surrounding the cavity from a source in the balloon. Determination of patient eligibility for APBI is made post-lumpectomy and is based on many factors, including the tumor margin status, nodal involvement, device compliance to lumpectomy cavity, device symmetry, balloon integrity, and proximity of the balloon surface to skin or chest wall. Computed Tomography (CT) scanning of the breast in three dimensions is the only practical method today to assess some of these requirements, such as device compliance to cavity, device symmetry, balloon integrity, and proximity of the balloon surface to skin or chest wall.

If the criteria are met, radiation therapy is typically provided by inserting ¹⁹²Ir seeds attached to a high-dose rate (HDR) remote afterloader into the inflated balloon for a short duration, typically less than 10 minutes. Typically, two treatments are administered per day, for a total of 5 days, to deliver the total prescribed radiation dose, usually 34 Gy. CT scanning or other imaging modalities such as surface ultrasound or X-ray imaging are used multiple times during the course of radiation therapy to assure continued device integrity, symmetry, position, diameter and conformance. Radiation therapy using a miniature x-ray source is also in development. After radiation therapy is concluded, the balloon is deflated and removed from the breast, often requiring intravenous pain killers.

While necessary, CT scanning of the breast to evaluate APBI treatment has certain drawbacks and shortcomings. First of all, CT does not always provide true three-dimensional visualization of the breast, the surrounding tissues, and the balloon. CT scanning creates successive 1-2 mm thick, transverse two-dimensional images, and displays them in a contiguous fashion. Structures or features smaller than 1 mm can easily be missed using CT alone. Another serious disadvantage of CT scanning is that it uses ionizing radiation to create these images. Though, the amount of radiation absorbed from CT scanning is very small, deleterious effects of ionizing radiation are well documented. Additionally, CT scanning is expensive and has to be performed within shielded rooms using expensive capital equipment operated by highly trained technicians and nurses. It is also not possible to use CT scanning easily in a peri-operative setting, which makes interactive use of CT impractical. Further, the balloon catheter must be filled with a mixture of saline and a radiographic contrast to provide a well defined balloon-tissue boundary for CT scanning. Concentration of contrast can be 25% or more of the balloon volume, causing radiation absorption and attenuation to tissue by up to 10%, and sub-optimal dosing of tissue volume. Radiographic contrast, though widely used, could also be expensive, especially in the developing countries. Finally there are risks and morbidity concerns associated with possible contrast leaks into the lumpectomy cavity.

If a patient is determined to be ineligible for APBI, the balloon catheter is removed and the patient is typically treated using conventional External-Beam Radiation Therapy (EBRT). Factors that might cause a patient to be ineligible include, positive tumor margin status, nodal metastasis, less-than-minimum required skin to source distance, less-than-minimum required chest wall to source distance, compromised balloon integrity, poor balloon symmetry, and poor balloon/tissue compliance. Even before the CT scanning is ever performed, the pathological examination of the excised tissue may identify additional disease left behind. As described earlier, this situation may occur in over 30% of the cases, requiring the balloon catheter to be removed and discarded and more tissue be excised.

Regardless of the reasons, delay in making eligibility decisions for the patients, causes anxiety as well as places the patient under additional infection risk, due to a balloon catheter having been installed in her lumpectomy cavity. Decisions of ineligibility cause despair and disappointment. Patient convenience, logistics, time, cost and morbidity associated with delay in making eligibly decisions limit the adoption of APBI today.

Recent clinical studies have shown infection risks of 16% or more using balloon catheter based APBI. The infection may cause pain, abscess and cellulitis and may require to be treated by oral or intravenous antibiotics, needle or incision drainage or surgical intervention. In some cases, additional infected tissue may have to be surgically removed.

Studies have also shown that about 25% of the patients develop mild acute skin reactions, which typically resolve over time. Though this is a significant improvement compared the EBRT results, it could be prevented by carefully reducing the radiation dose to the surrounding skin. Long term data is not yet available for APBI. However heart and lung complications and fat necrosis and other late occurring side effects may eventually be experienced.

Removal of device in most cases is preceded by intravenous injection of a mild analgesic, such as ibuprofen, acetaminophen/codeine or lorazepam. In rare instances, the balloon may stick to the breast tissue, making removal from the cavity more difficult.

For these reasons, it would be desirable to eliminate the need to employ CT imaging when using a catheter to treat a lumpectomy or other body cavity. It would be particularly desirable to have a device that could peri-operatively characterize the lumpectomy or other body cavity and detect additional disease that might be present. Local recurrence (recurring tumor around the original tumor site) of breast cancer is believed to be related to original microscopic disease left behind during the surgical procedure as well as the quality of the radiation therapy delivered. Detection and removal of additional disease at the original lumpectomy procedure might have profound consequences such as reduced possibility of local recurrence. Such detection and removal of additional disease could also reduce the number of re-excisions that are performed because not enough tissue was removed during the first lumpectomy procedure.

To overcome these shortcomings of CT scanning, it would be desirable to provide a three-dimensional imaging or other detection modality that can more accurately confirm integrity, proximity, and conformance to breast tissue of the balloon or other radiation delivery component. An imaging modality should also be convenient and economical, take less time and effort to perform, and pose little or no morbidity risks to the patient. The new imaging modality should also be usable at the time of original lumpectomy. In addition to all the other advantages mentioned, achieving optimal compliance of the balloon and breast tissue in the operating room may be possible by visualization and small adjustments of the actual balloon within the lumpectomy cavity.

A further objective of the present invention would be to allow patient eligibility decisions earlier in the chain of patient care, preferably at the time of lumpectomy, not several weeks later. This improvement would eliminate patient disappointment, reduce infection rates as well as cost of medical products installed but never used and additional medical procedures performed.

2. Description of the Background Art

Systems and methods for delivering radiation to treat body cavities are described in U.S. Pat. Nos. 5,913,813; 6,074,338; 6,095,966; 6,413,204; and US Patent Application 2005/0240073. Systems and methods for ultrasonic imaging through a balloon are described in U.S. Pat. Nos. 6,142,945; 6,004,273; and 5,335,663, and in US2006/000968.

### SUMMARY OF THE INVENTION

The present invention provides methods and apparatus for characterizing tissue surrounding a surgical resection cavity in solid tissue, such as a lumpectomy cavity formed in a breast following breast cancer surgery. The characterization may comprise imaging of the cavity tissue, such as ultrasonic imaging of the tissue, and may be used as an aid in planning subsequent treatment of the surrounding tissue. Optionally, the methods and apparatus may further provide for treatment of the surrounding tissue, such as by exposing the tissue to ionizing or non-ionizing radiation. Such characterization capability may also allow for inspection of the integrity and position of the balloon or other imaging or treatment component prior to any treatment.

In a first aspect of the present invention, a method for characterizing tissue surrounding a surgical resection cavity in solid tissue comprises introducing a tissue characterization device into an interior of the cavity and characterizing at least a portion of the surrounding tissue using the tissue characterization device. Typically, a tumor had been surgically resected from solid tissue to create the surgical resection cavity. Most commonly, the solid tissue is breast tissue which has been surgically treated to remove a tumor, leaving the resection cavity. The characterization device is usually positioned through a transcutaneous opening formed during the tissue resection surgery. Most commonly, the surgery will be open surgery so that the characterization device may be easily positioned in the cavity from which the tumor has been removed. In other cases, the characterization device may be introduced through a small transcutaneous opening formed in a minimally invasive procedure to remove the tumor.

Usually, the methods will further comprise initially positioning the distal end of an introducer catheter in the surgical resection cavity to provide access for the imaging or other characterization apparatus. The introducer catheter may at times be referred to as a balloon delivery catheter or radiation delivery apparatus hereinafter. The introducer catheter will include a lumen which allows advancement of the tissue characterization device through a catheter to the distal end within the cavity. The distal end of the introducer catheter will typically have an enlarged or enlargeable distal member or structure, such as an inflatable balloon or a fixed geometry structure. The inflatable balloon or other enlargeable structure may be expanded in situ within the cavity so that the balloon surface conforms closely to the surrounding tissue. Such close conformance is advantageous, both during the imaging or other characterization of the tissue and to help create a symmetric, regular shape for the cavity which helps assure subsequent treatment, particularly from ionizing radiation. The enlarged structures may be contracted, e.g. by deflating an inflated balloon to facilitate removing the introducer catheter from the surgical resection cavity at the end of treatment.

When the enlarged distal end has a fixed geometry, the size will typically be about equal to or slightly greater than the volume of tissue resection cavity. In this way, the fixed geometry structure will fit or slightly distend the cavity to help assure close conformance. Usually, the fixed diameter structure will be frangible, collapsible, or otherwise destructible so that it may be reduced in size prior to removing the introducer catheter from the surgical resection cavity.

In still other cases, the enlarged structure on the introducer catheter may have a biodegradable structure. Such biodegradable enlarged structures will typically be removably attached to an introducer shaft which will usually not be biodegradable, thus allowing removal of the shaft after the treatment is complete. The enlarged structure will then be left in place but will degrade in situ over a preselected period of time, typically weeks or months.

In the case of all such enlarged structures, a vacuum may be drawn in the interstitial region between the surface of the enlarged structure and the tissue to draw the tissue against an outer surface of the enlarged structure. Applying a vacuum to draw the tissue against the structure further promotes close conformance between the tissue and the enlarged structure, allowing for enhanced imaging and treatment.

The tissue may be characterized in a variety of ways, including thermal mapping, histochemical staining, biochemical analysis, or a variety of other techniques. Preferably, however, the tissue will be characterized by imaging, particularly by ultrasonic imaging or by the closely related optical coherence tomography (OCT). By imaging the tissue, residual calcification can be detected to indicate that some tumor cells may have been left behind in the original surgical resection. In such instances, it is possible to immediately reopen the cavity and surgically remove additional tissue before the patient has left the operating room. In a particular preferred aspect of the present invention, three-dimensional images may be obtained which allow for improved detection of residual tumor cells. In addition to detecting residual disease, imaging within the introducer catheter can help confirm that the tissue closely conforms to the enlarged structure and further to detect if any leaks or structural breaches have occurred in the introducer catheter. In a specific embodiment, the tissue characterization device comprises one or more ultrasonic transducers, where the transducers are rotationally and/or axially scanned or with enough transducers deployed such that they remain stationary within the distal end of the introduced catheter, optionally producing a three-dimensional image of the cavity tissue.

In addition to characterizing the tissue in the resection cavity, the methods of the present invention may further comprise treating at least a portion of the tissue surrounding the resection cavity. Such treatment will typically comprise delivering ionizing or non-ionizing radiation to the tissue, particularly to treat residual cancer cells which may remain. Treating the tissue surrounding the surgical resection cavity typically comprises positioning a treatment device through the lumen of the introducer catheter, typically by exchanging the treatment device with the characterization device.

In a second aspect of the present invention, a method for characterizing and treating tissues surrounding a surgical resection cavity comprises positioning an introducer catheter transcutaneously so that a distal end of the catheter is positioned in the surgical resection cavity and a proximal end of the catheter is external to the tissue. The tissue characterization device is positioned through the introducer catheter and at least a portion of the tissue surrounding the surgical resection cavity is characterized using the tissue characterization device. After the tissue has been characterized, the tissue characterization device is typically removed from the introducer catheter, and a tissue treatment device is introduced through the introducer catheter. At least a portion of the tissue surrounding the surgical resection cavity is treated using the tissue treatment device. In most cases, the information obtained by characterizing the tissue allows the treatment to be directed at particular diseased regions of the tissue, such as residual tumor cells when treating a tissue cancer. Particular features of this second aspect of the present invention are the same as described previously in connection with the first aspect of the present invention.

In a third aspect, the present invention comprises a system for characterizing and/or treating tissue in a surgical resection cavity. The system comprises an introducer catheter including a shaft having a proximal end, a distal end, a central lumen therethrough, and a distal structure positionable in the surgical resection cavity. The system further comprises at least one tissue characterization or tissue treatment element insertable through the central lumen and having an active component which is positionable within the distal structure.

The system may comprise at least one tissue characterization element adapted to detect residual tumor cells in the cavity wall tissue, for example comprising an ultrasonic imaging transducer or array. Any of the other characterization devices mentioned previously in connection with the methods of the present invention may also be incorporated into the systems.

The system may comprise at least one tissue treatment element adapted to treat residual tumor cells in the cavity wall tissue, for example comprising an ionizing or non-ionizing radiation source. Other treatment elements include radiofrequency electrodes, cryogenic delivery systems, heat, high intensity focused ultrasound (HIFU) and the like.

In a preferred embodiment, the systems of the present invention will comprise at least one tissue characterization device and at least one tissue treatment device, in combination. The nature of the tissue characterization device and tissue treatment device are the same as described above.

The distal structure of the introducer catheter will typically be enlarged or enlargeable. For example, the distal structure may comprise a balloon or other inflatable and deflatable structure. Alternatively, the distal structure may have a fixed geometry which is introduced in its enlarged size to the tissue cavity. After the tissue characterization and/or treatment is completed, the fixed geometry distal structure may be collapsed, broken down, or otherwise reduced to a smaller size to facilitate removal from the cavity. As a third alternative, the distal structure may be biodegradable so that it need not be removed after the characterization and treatment protocols are completed.

### BRIEF DESCRIPTION OF THE DRAWINGS

FIG. 1 illustrates an interstitial balloon catheter used for both ultrasound imaging and delivery of radiation therapy to the interstitial cavity.

FIG. 2 illustrates an ultrasound imaging probe positioned in a balloon catheter.

FIG. 3 illustrates a radiation therapy delivery device positioned in a balloon catheter.

FIG. 4 is a flowchart illustrating the steps in a treatment protocol in accordance with the principles of the present invention.

FIG. 5 is a flowchart illustrating the steps in an imaging protocol using an ultrasound probe in a balloon catheter in accordance with the principles of the present invention.

FIG. 6 is a flowchart illustrating the steps in a radiation therapy protocol using a radiation source positioned in a balloon catheter in accordance with the principles of the present invention.

FIGS. 7A and 7B illustrate an embodiment of an introducer catheter having a fixed geometry shown both in an enlarged configuration (FIG. 7A) and in a collapsed configuration (FIG. 7B).

FIGS. 8A-8C illustrate an introducer catheter having a biodegradable enlarged structure at its distal end. The catheter comprises at least a shaft structure (FIG. 8B) and a removable, biodegradable enlarged structure (FIG. 8C).

FIGS. 9A-9B illustrate alternative embodiments for ultrasonic imaging tissue characterization devices which may be used in the systems and methods of the present invention.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, representative methods, devices and materials are now described. Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein are incorporated herein by reference for the purpose of describing and disclosing the components that are described in the publications which might be used in connection with the presently described invention.

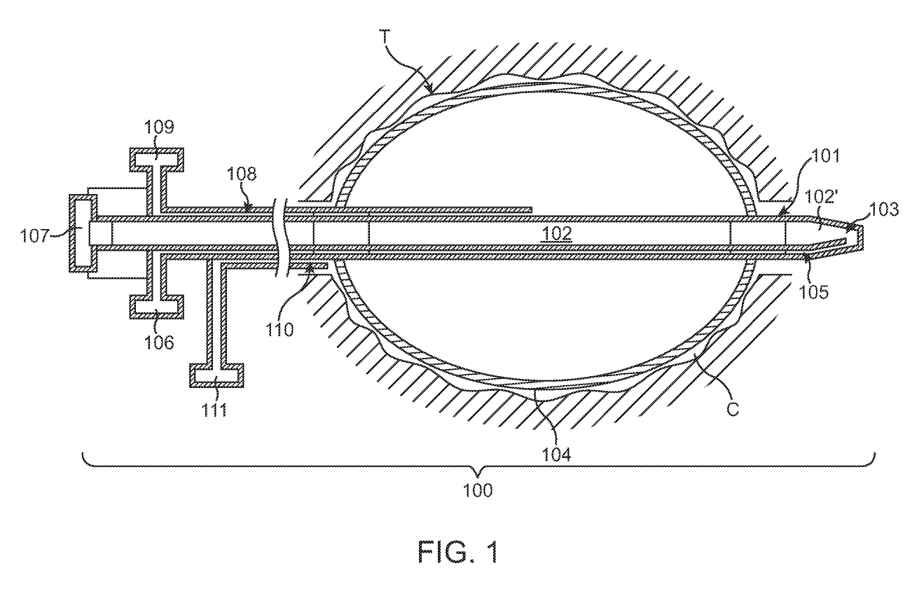

Referring to FIG. 1, a balloon catheter 100 suitable for placement in a body cavity, such as at site of tumor resection, includes an inflatable balloon member 104, referred to hereafter as a “balloon.” The balloon is typically composed of an elastic, compliant material, such as silicone, nylon, polyethylene, etc. The balloon shape may be spherical, elliptical, substantially elliptical, spiral, or the like. Alternatively, the balloon may comprise a plurality of small balloons or other inflatable structures to produce an overall generally spherical or elliptical (ovoidal) shape. Such shapes provide optimal conformity/flexibility to surrounding tissue during placement of the device. Preferably, the balloon is shaped to completely occupy the surgical resection cavity, leaving a minimum size and number of surrounding air pockets. The balloon surface comprises at least a single layer, optionally comprising two or more layers and still further optionally, being laminated. In the preferred embodiment the balloon member 104 has a width or diameter in the range from approximately 2-4 centimeters. However, depending on the specific requirements for a cavity to be treated, the balloon member 104 could be designed larger or smaller.

Other configurations may also be used. For example, the balloon surface may have a bi-layer structure comprising an internal solid layer, and an external, porous layer. The balloon surface may also comprise folds, ridges, or bars (trabeculae). These features may be useful in drawing the tissue against the balloon to achieve optimum tissue conformance, good quality and complete ultrasound imaging, and for removal of seroma produced by the body. Additionally, therapeutic agents, drugs, painkillers, etc. may be carried by the external, porous layer for delivery to the surgical cavity.

The balloon catheter 100 has an elongate shaft 101, having at least one central lumen 102 that terminates distally in one or more fluid communication ports 103. For clarity, only one distal fluid communication port is illustrated in FIG. 1. The fluid communication port 103 is positioned near to the distal tip of the catheter, shaft 101 beyond a distal end of balloon member 104 to ensure that all air in central lumen 102 is pushed forward and into fluid return channel 105. Though it is not shown in FIG. 1, the port 103 could also be in fluid communication with the interstitium through an opening (not shown) at the distal end of shaft 101. Channel 105 is formed within the walls of the catheter, and is in fluid communication with and accessible via fluid return port 106 near the proximal end of the catheter. Central lumen 102 is accessible via central lumen port 107 at the proximal end of the catheter. The lumen 102 of the catheter 100 can be used for first delivering the ultrasound or other tissue characterization catheter and subsequently a radiation delivery device, as described in more detail below.

A balloon inflation lumen 108 is also formed within the walls of the catheter and communicates with the interior of balloon member 104 and is used to inflate/deflate the balloon via balloon inflation port 109 near the proximal end of the catheter. The space comprising lumen 102 and channel 105 does not communicate with the space defined by balloon inflation lumen 108.

An interstitial communication channel 110 is also formed within the walls of the catheter and communicates with the interstitium and is used for suction of the tissue or seroma, or for delivery of fluids or agents in to the interstitium. Suction and/or fluid or agent delivery may be made through an interstitial communication port 111 near the proximal end of the catheter 100. Application of negative pressure at port 111 allows for aspiration of post-surgical drainage and/or tissue conformance/collapse onto the balloon surface. Fluid or agent injection (e.g. delivery of medications, hormones, agents, proteins, cells, etc.) into the interstitium over the surface of the balloon member 104 is also possible through the port 111.

The central lumen 102 of catheter 100 should be purged of air for optimum ultrasound imaging. Central lumen port 107 may comprise a Touhy-Borst valve assembly or other three-way-connectors. Lumen 102 communicates with the fluid return channel 105 via a tapered segment 102′ that leads to the internal fluid communication port 103. Channel 105 terminates in the fluid return port 106.

The catheter system of FIG. 1 provides a closed circulation system which is not connected to the interior of the balloon member 104. This system permits filling and flushing of catheter shaft central lumen 102 with a liquid, such as saline, oil, etc. while allowing air to purge through fluid return channel 105. The tapered segment 102′ of central lumen 102 at the junction with channel 105 maximizes air purge from the central channel. Air purging improves ultrasound imaging from within catheter shaft. Other means for facilitation of air purge, such as modified core channel shapes, internal valves, multiple evacuation orifices, and channels could also find use.

The devices employed in the subject invention, particularly the catheter shaft 101 and the balloon member 104, should be made at least partly of materials transparent to the imaging signals, such as ultrasound. Accordingly, the devices may be made of variety of plastics, such as silicone, polyethylene, PFE, PTFE and the like.

In use, the balloon catheter 100 is first inserted into the body cavity with the balloon member 104 in a deflated state. A fluid such as saline is introduced through the balloon inflation port 109 and lumen 108 to inflate the balloon. While the balloon member 104 remains inflated, another fluid, such as saline, is infused through the closed flow path defined by port 107, central lumen 102, fluid communication port 103, fluid return channel 105 and fluid return port 106, purging air out of the central lumen 102 in preparation for ultrasound or other imaging. A vacuum may also be applied at the interstitial communication port 111 to remove fluid and gases surrounding the inflated balloon and draw tissue T in the surrounding cavity C closer to the outer surface of the balloon. Alternatively or additionally, the port 111 could be used for evacuating seroma out of the body or for infusing therapeutic agents into the body cavity C.

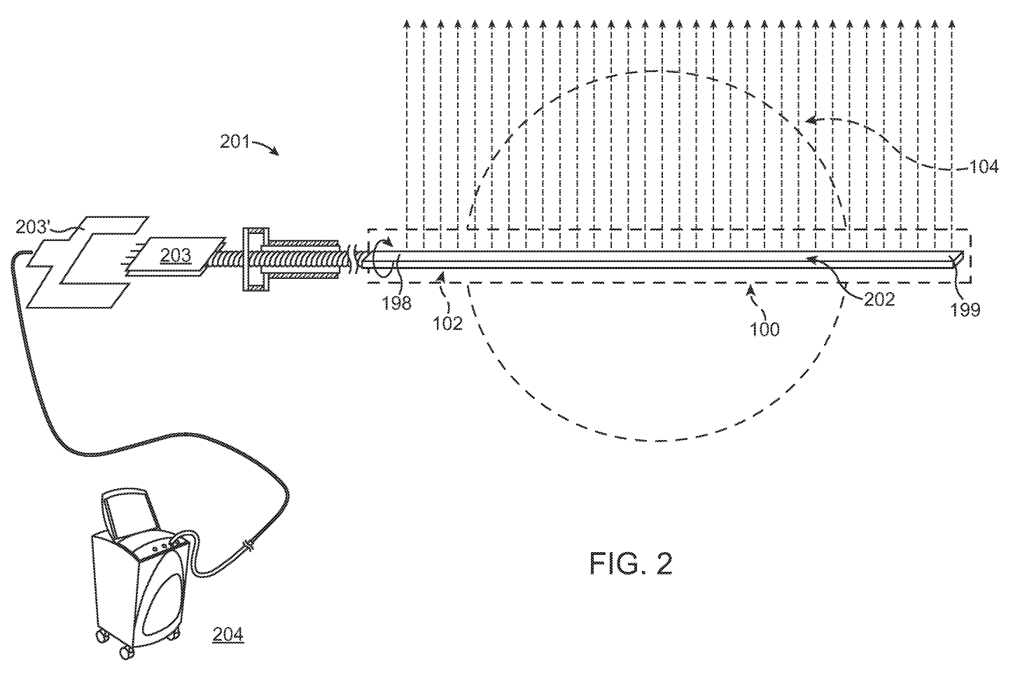

FIG. 2 shows an ultrasound imaging probe 201, in the form of a linear array 202, slideably positioned inside the central lumen 102 of balloon catheter 100. The ultrasound probe 201 can be an integral part of the balloon catheter 100, or more often will comprise a separate device designed for introduction into lumen 102, as shown in FIG. 2. The ultrasound probe 201 may include a flexible elongate shaft, but may also be rigid in its distal section. Flexible, elongate ultrasound probes are well known in the art. It is anticipated that in the operating room the probe 201 would be exposed to blood and other bodily fluids. In order to keep the probe from such contamination, the probe 201 could first be inserted in a disposable outer sheath 205 (not shown) and then slipped into lumen 102. The configuration and movement of the probe 201 determines the directions in which the imaging beam will be directed. The probe 201 consists of a proximal end 198 and a distal end 199. The linear transducer array 202 is connected via a proximal connector 203 and coupler 203′ to an ultrasound imaging apparatus 204 for sending high frequency pulses to the body cavity for receiving, constructing and analyzing ultrasound signals. The apparatus 204 can interface with a radiation treatment planning system to better tailor radiation therapy by helping to determine dwell time and dwell locations of the radiation delivery device, based on the interpretation of the imaging results. The ultrasound apparatus 204 and radiation treatment planning system 304 could share the same computer, could be installed in one integrated system or could be two separate devices.

An exemplary linear transducer array 202 is approximately 70 mm in length, operates at or around 10 MHz, with a pitch of about 230 microns, thus containing about 300 elements. The elevation of this linear array will be around 4 mm. The linear transducer array 202 is side viewing and produces a rectangular field of view. When rotated, the array 102 will generate a cylindrical volume of ultrasound images of the balloon catheter 100 and the surrounding tissue around the long axis of the catheter 100. Rotation of the entire probe 201 with the array 202 may be achieved by a motor driven mechanism, or a spring-loaded actuator, etc. Rotation usually includes at least one complete rotation about the longitudinal axis of the catheter 100 or may comprise a continuous rotation until stopped. Additionally, the probe 201 may be pulled linearly along the length of the catheter 100, lengthening the cylindrical volume generated by the amount of pullback. Pulling back an ultrasound probe to generate 3D images is well known in the art. The device of the invention can provide an ultrasound image depth of penetration beyond 35 mm, thus creating images of structures beyond the outer layer of the balloon member 104 or the shaft 101. At 35 mm, each echo takes 45 μsec to make the round trip, and if 256 image lines are used to generate a complete image, it will take about 12 msec to generate a complete image, thus providing real time imaging. If a frame takes less than 33 msec to be generated, it is considered to be “real time.”

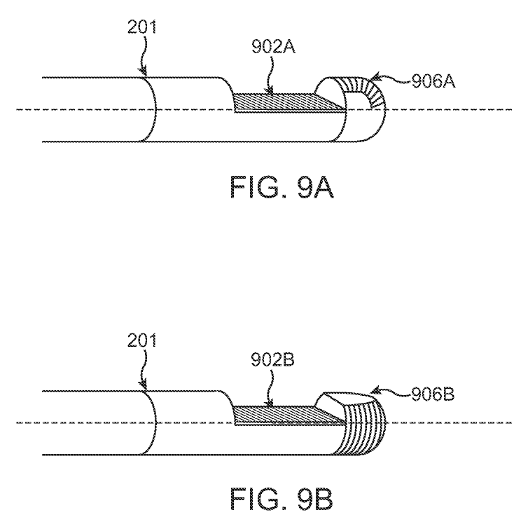

Other embodiments of ultrasound imaging probe 201 are illustrated in FIGS. 9A and 9B where a side-viewing linear array is placed next to a forward viewing curved array. In FIG. 9A, the forward viewing curved array 906A is a continuation of a side-viewing linear array 902A. In FIG. 9B, a forward viewing curved array 906B has an orientation that is perpendicular to a side-viewing linear array 902B. If the probe of FIG. 9A is used, a rotation of 360 degrees about the long axis of the catheter would be needed to generate a complete 3D volume in the shape of a cylinder with a hemispherical dome. If the probe of FIG. 9B is used, the forward hemispherical dome would have double coverage, allowing image averaging to improve the image quality. A half length curved array with 360 degree rotation or a full length curved array with 180 degree rotation are other approaches to generate a complete hemispherical coverage.

While specific acoustic parameters have been provided in this description, many variations are possible and would be obvious to one skilled in the art. Similarly, various matching and backing layers used in transducer construction to improve the imaging performance, as well as use of an acoustic lens for a fixed focus in the elevation dimension are envisioned and are well known in transducer design prior arts. It will also be possible to anticipate to one skilled in the art that an ultrasound image of the body cavity need not be generated in all circumstances. The radiofrequency (RF) signature of the return signal from the body cavity may contain diagnostic information. Observation and analysis of this signature may suffice to guide possible subsequent surgical resection and or radiation therapy.

The ultrasound probe 201 could be disposable or non-disposable, could be re-sterilized after each use, or could be saved through the radiation therapy for each patient and replaced between patients in which case an outer sheath 205 may not be required.

One skilled in the art would appreciate that the above embodiment would require over 300 wires to traverse the length of the catheter 100 and attach to individual elements of the linear array transducer 202. In one embodiment, a multiplexer is used to time share a smaller number of active channels. At each transmission, only a small set of transducer elements is used and a plurality of sequential transmissions is used to activate all elements and create a complete image. The shallow imaging depth allows this plurality of transmissions to take place within a relatively short time such that real time imaging remains possible. If one considers an example where a 16:1 multiplexer is used, then about 16 transmissions can be used to generate one image line taking about 720 microseconds. For an image frame having 256 image lines, it would require about 185 msec, no longer fast enough for a real time display through may be adequate for objects that are by and large static. Use of multiplexer can significantly reduce size, cost and weight of the catheter due to the reduction of the number of conductor channels that would otherwise be needed.

It is also possible to use the linear array transducer 202 to deliver acoustic energy to break up larger air bubbles which may be present in the tissue cavity surrounding the balloon member 104 to smaller ones which can be removed with the application of vacuum at the interstitial communication port 111. Removing such surrounding air bubble provides for better quality ultrasound images by achieving better conformance of the tissue to the balloon surface. The acoustic field parameters that can be modified for this purpose include frequency, duty cycle, excitation waveform, power and focusing, among others.

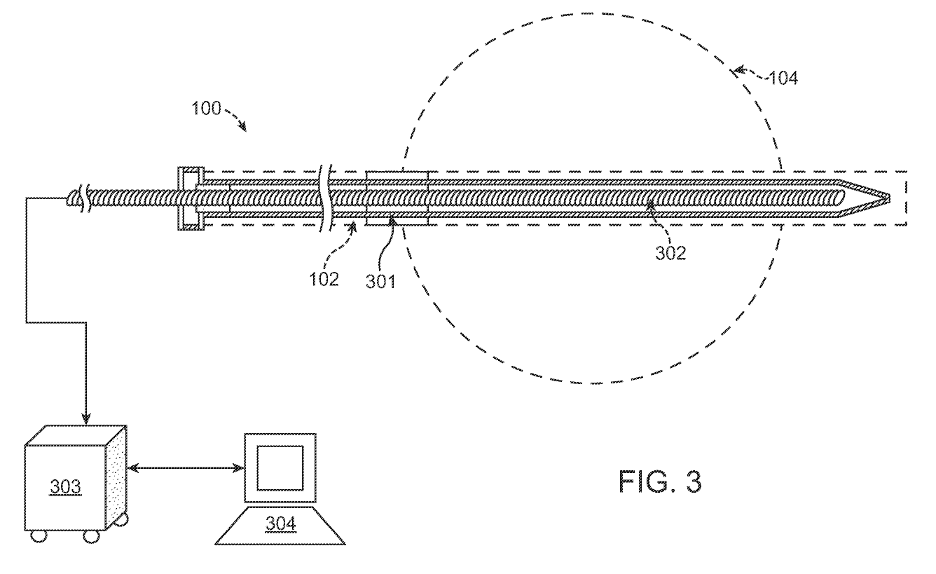

As shown in FIG. 3, the balloon catheter 100 may also be used to deliver radiation therapy using a radiation guide tube 301 slideably received in the central lumen 102 of catheter 100. The radiation guide tube 301 is inserted into central lumen 102 through the port 107 (FIG. 1). A radiation source 302 is then inserted into the guide tube 301, and radiation therapy may be delivered by an external radiation delivery apparatus 303 according to the radiation treatment plan devised by the radiation treatment planning system 304.

The radiation source 302 of the present invention can be a radioactive wire, a radioactive seed, a radioactive fluid filled delivery system, a miniature X-ray tube or the like, or non-ionizing radiation sources, such as a probe containing a high intensity focused ultrasound (HIFU) transducer. The nature of the external radiation delivery apparatus 303 will depend on the nature of the radiation source 302. In the case of an X-ray source, the apparatus 303 will include the electronics necessary to power the X-ray source.

The radiation planning system 304 can utilize the information generated by the ultrasound imaging apparatus 204 to derive the radiation treatment plan. The radiation treatment planning system 304 and the ultrasound imaging apparatus 204 could be integrated into one same system.

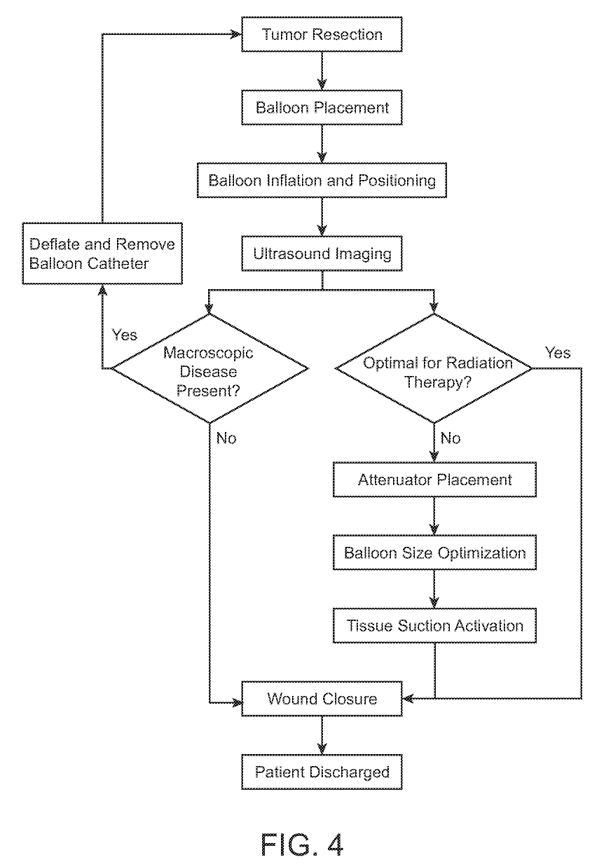

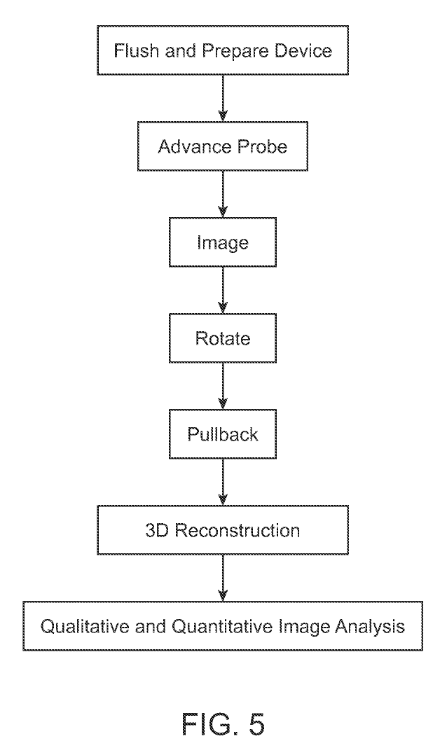

A breast or other solid tissue tumor may be treated by initially resecting the tumor to create a cavity C in the tissue T, as shown in FIG. 1. After tumor resection, the balloon catheter device 100 is placed in the cavity C and used to characterize and optionally treat the cavity peripheral tissue as set forth in FIG. 4. Initially, the catheter 100 is introduced with the balloon member 104 deflated. A fluid, such as saline, is infused through the balloon inflation port 109 to inflate the balloon member 104 to its second, inflated shape. Balloon catheter device 100 is then further positioned within the body cavity. After preparing, positioning and using the ultrasound probe in a manner described in FIG. 5, an ultrasound image is generated and interpreted for evaluation of any macroscopic disease left behind or for whether optimal radiation therapy can be delivered with the current balloon catheter 100 in its present position or shape.

If the image analysis shows that a significant residual tumor is still present, the catheter 100 is deflated and temporarily removed from the body cavity, and the surgical tumor excision may be repeated until a satisfactory result (as determined by imaging as just described) is obtained. Additional resection may be followed by the same steps of balloon placement, inflation and positioning and ultrasound imaging. At any time during the operation, the physician may determine the patient to be ineligible for subsequent radiation or other balloon-based treatment.

If the image analysis shows that radiation therapy is appropriate, but some radiation vulnerable or healthy tissue is too close to the radiation source, subcutaneous attenuation devices may be placed between the catheter 100 and such tissue to prevent radiation damage to such tissue. The balloon member 104 size may be optimized, if necessary, to make the areas of desired exposure and desired shielding within the proper distance from the radiation source to provide the desired dose. If the balloon member 104 includes tissue conformance features, such as surface ridges, suction may be applied through the port 111 to achieve better tissue conformance with the balloon member 104. At any time during the operation, the physician may determine the patient to be ineligible for the treatment procedure and remove the balloon catheter 100 from the cavity.

The wound and the incision are then closed with the device 100 in place. At this point, the ultrasound probe 201 may be removed from the central lumen 102 of the balloon catheter 100. The balloon catheter 100 however, is left behind to enable subsequent radiation therapy as part of the continued care. The patient is discharged to take any necessary medications and for healing of the original excision site. The patient is brought to the hospital, typically in about one week, to receive radiation therapy according to a schedule determined during radiation treatment planning.

Imaging may be performed by activating the ultrasound imaging apparatus 204. Under the control of the ultrasound apparatus 204, the probe 201 is rotated and may be pulled back, to create a three dimensional (3D) ultrasound image of the surrounding structures. These 3D images are then examined for confirmation of disease status in the cavity as well as balloon and tissue conformance, distance of the device to skin and other sensitive organs.

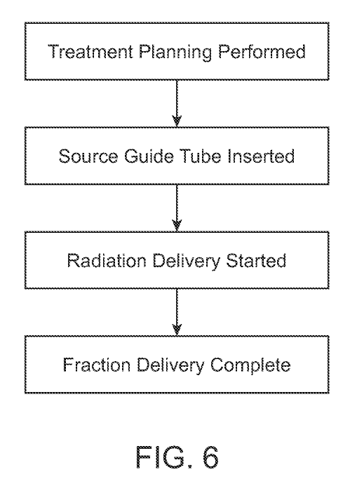

As shown in FIG. 6, the steps for preparing and delivering the radiation therapy device 302 according the invention includes analyzing ultrasound images obtained during surgical excision. Treatment planning is performed to determine dwell positions, dwell times as well as dose(s) to be delivered to the tissue surrounding the body cavity C. After the patient is admitted for the radiation therapy portion of her care, the source guide tube 302 is inserted into the central lumen 102 of catheter 100. Under the guidance and direction of radiation treatment planning system 304, the radiation delivery apparatus 303 is used to advance and control the radiation delivery device 302 through source guide tube 301, and radiation is delivered to the body cavity C and surrounding tissues T. After each radiation therapy fraction has been delivered according to the treatment plan, the radiation delivery device 302 is removed from the source guide tube 301. The guide tube 301 may also be removed from catheter 100. The catheter 100, however, is left in place to receive the next fractionated dose, usually within 6 hours.

Prior to such radiation treatment, ultrasound imaging may optionally be performed as described above (FIG. 5) to ensure the balloon catheter has not moved, been punctured, or changed its geometry or size. Additional diagnostic information such as presence of air pockets, seroma, and/or proximity of healthy tissues to the radiation source can be determined and such information could be used to modify the radiation treatment regimen going forward.

The system of the invention is useful as an imaging catheter to provide images of the inflated balloon and the surrounding tissues. If ultrasound energy is utilized, the ultrasound images are advantageous in determining the dimensions of inflated balloon, assessing ultrasonic tissue characteristics and topographical relationships of the surrounding tissue, determining position and distance of radiation-vulnerable tissues from the radiation source, and estimating dosage, dwell location and times for subsequent radiation treatment planning.

As described thus far, the catheter has included an inflatable member, typically a compliant balloon. In alternate embodiments, the catheter may comprise a relatively rigid distal structure for surrounding the imaging and/or treatment devices. Preferably, the rigid structure will be collapsible to facilitate removal of the catheter at the end of the treatment. For example, the catheter may comprise a distal member formed as a generally spherical thin wall, collapsible, polymeric material that in its uncollapsed shape, resembles a spherical or ovoid ping-pong ball. Such an apparatus would be useful in insertion into the surgical cavity during open, invasive surgery, in its uncollapsed shape. The apparatus can later be removed through a small incision in the skin after collapsing the structure into its collapsed, smaller, second shape by, for example, use of vacuum, suction, mechanical crushing, folding or any other means, The transition of the distal member shape from the first, un-collapsed shape to the second collapsed shape is irreversible.

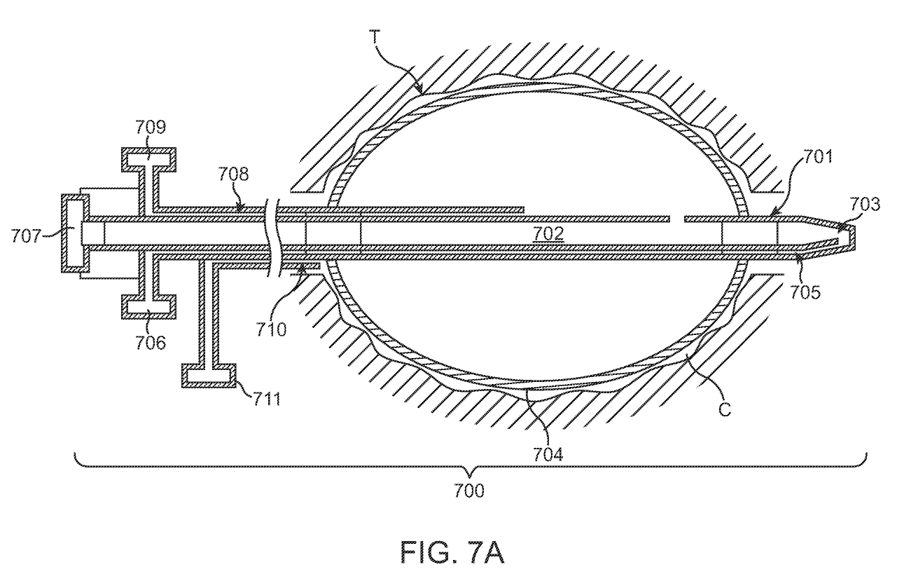

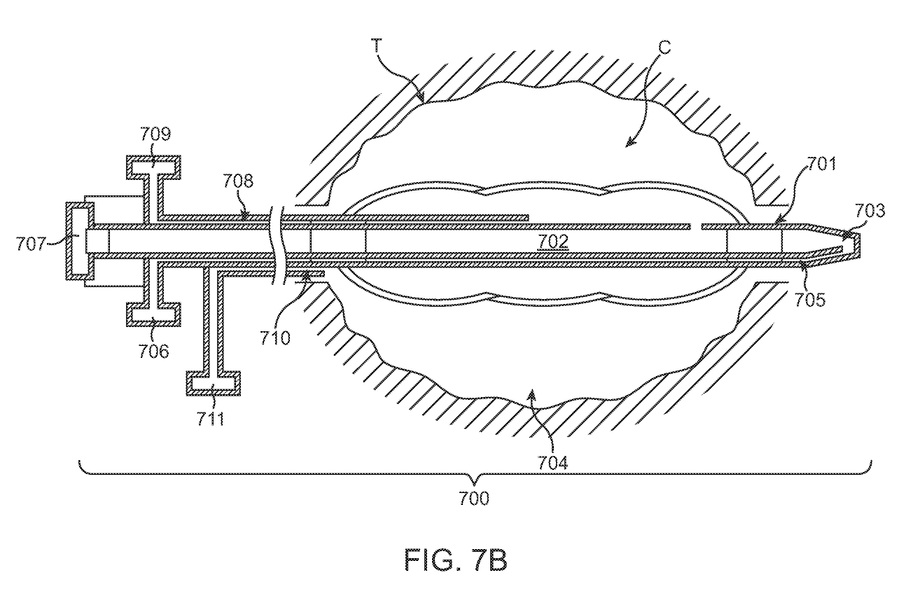

Referring to FIGS. 7A and 7B, a collapsible interstitial tissue/characterization/radiation delivery apparatus 700 is adapted for placement in a body cavity C, typically a site of tumor resection in solid tissue T. A distal member 704 of the apparatus 700 is collapsible, typically made of non-elastic, noncompliant material. The distal member shape may be spherical, elliptical, substantially elliptical, or spiral, or comprise a plurality of small distal members to produce an overall generally spherical or elliptical shape. Such shapes provide optimal conformity/flexibility to surrounding tissue during placement of the device. Typically, the shape of the distal member conforms closely to the shape of the surgical resection cavity C, leaving a minimum volume and number of air pockets surrounding the member 704. As shown, the distal member 704 comprises at least a single layer, typically having a diameter of approximately 2-4 centimeters. Depending on the specific requirements for a cavity to be treated, however, the distal member 704 could be larger or smaller.

Other configurations may also be used. For example, the distal member surface may have a bi-layer structure comprising an internal solid layer, and an external, porous layer. The distal member surface may also comprise folds, ridges, or bars (trabeculae). These features may be useful in drawing or sucking the tissue against the distal member to achieve optimum tissue conformance, good quality and complete ultrasound imaging and for removal of seroma produced by the body. Additionally, therapeutic agents, drugs, painkillers, etc. may be delivered through this feature to the surgical cavity.

The apparatus 700 is otherwise constructed similarly to catheter 100, and has an elongate shaft 701, having at least one central lumen 702 that terminates distally in one or more fluid communication ports 703. For clarity, only one distal fluid communication port is illustrated in FIG. 7A. The fluid communication port 703 is positioned near to the distal tip of the apparatus to help ensure that most or all the air in the central lumen 702 is pushed forward and into fluid return channel 705. Though it is not shown in FIG. 7A, the port 703 could also be in fluid communication with the interstitium (space surrounding the member 704) with an opening at the distal end of shaft 701. Channel 705 is formed within the walls of the apparatus 700 and is in fluid communication with return fluid port 706 near the proximal end of the apparatus. The central lumen 702 is accessible through central lumen port 707 at the proximal end of the apparatus. The central lumen 702 is used for first delivering the ultrasound catheter and consequently a radiation delivery device, as with catheter 100.

A distal member fluid/suction lumen 708 is formed within the walls of the apparatus 700 and communicates with the interior of distal member 704 and is used to fill the distal member with an ultrasound imaging compatible fluid, such as saline or contrast, during imaging. Additionally, the fluid/suction lumen 708 is used to evacuate all fluid from the distal member 704 and may be used to collapse the distal member into its second, collapsed shape by applying suction through the distal member fluid/suction port 709 near the proximal end of the catheter, while blocking ports 706 and 707. The space comprising lumen 702 and channel 705 must communicate with the space defined by distal member fluid/suction lumen 708 and the distal member 704, to ensure no air pockets are present during ultrasound imaging.

Interstitial communication channel 710, also formed within the walls of the apparatus, communicates with the interstitium and is used for suction of the tissue or seroma, or for delivery of fluids or agents into the interstitium via interstitial communication port 711 near the proximal end of the apparatus 700. Application of negative pressure at port 711 allows for suction for post-surgical drainage, or tissue conformance/collapse onto the distal member surface. Fluid or agent injection (e.g. delivery of medications, hormones, agents, proteins, cells, etc.) into the interstitium is also possible through the port 711.

The fluid communication lumen 702 of apparatus 700 must be purged of air for optimum ultrasound imaging. Central lumen port 707 may comprise a Touhy-Borst valve assembly or other three-way connectors. Lumen 702 communicates with the fluid return channel 705 via a tapered segment that leads to the internal fluid communication port 703. Channel 705 terminates in external fluid return port 706.

The apparatus 700 is inserted into the body cavity with the distal member 704 in its uncollapsed state. A fluid such as saline is infused through the fluid/suction port 709 to create an ultrasound imaging compatible environment. Another fluid, such as saline, is infused through the port 707, traveling through lumen 702, fluid communication port 703, fluid return channel 705 and out the fluid return port 706, purging the air out of the central lumen 702. A vacuum may be applied at the interstitial communication port 711 to pull the tissue closer to the distal member. Similarly, the port 711 could be used for evacuating seroma out of the body or for infusing therapeutic agents into the body cavity. As illustrated in FIG. 7B, upon completion of the treatment, the distal member 704 is collapsed and the apparatus 700 is pulled out of the body.

Although not shown in any of the figures, it is also possible to create yet a simpler apparatus by eliminating port 709 altogether and performing the fluid filling and suction tasks via ports 706 or 707, since ports 706 and 707 and the distal member 704 are in fluid communication with one another.

In alternate embodiments, a distal member may comprise a generally spherical, ovoidal, spiral, or other thin wall, bioresorbable implant. The bioresorbable material may be mostly gelatinous, or polymeric, such as polyglycolic acid, polyglactin, poliglecaprone or other synthetic absorbable elastomer. A cross-linking agent, such as glutaraldehyde, may be used to harden the material to desired consistency during manufacturing. Such an apparatus would be useful in insertion into the surgical cavity during open, invasive surgery. In addition to helping to deliver the post surgical radiation to the lumpectomy cavity in a uniform, homogenous manner, the bioresorbable implant could be used as a transport mechanism to deliver therapeutic agents, such as chemotherapy drugs, antibiotics, hormones, etc. and could be used for placing radio opaque markers in and around the cavity for future diagnosis and interventions. Additionally, the implant could create better cosmetic results as it would allow the tissue growth in the cavity to be more homogenous and gradual. A detachable shaft will usually be coupled to the implant through the coupling means and a septum, and used only during ultrasound imaging and the radiation therapy. After the conclusion of radiation therapy, the shaft is detached from the apparatus and discarded. The implant left in place is slowly resorbed by the surrounding tissue over a course of several months.

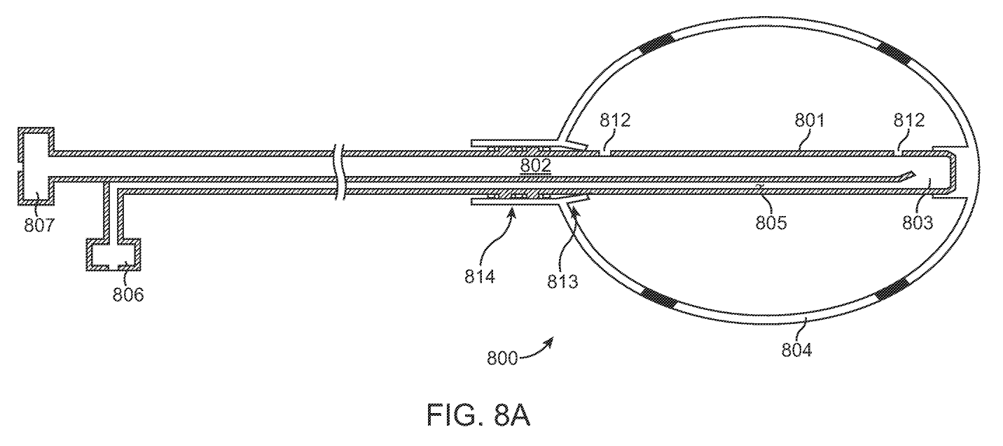

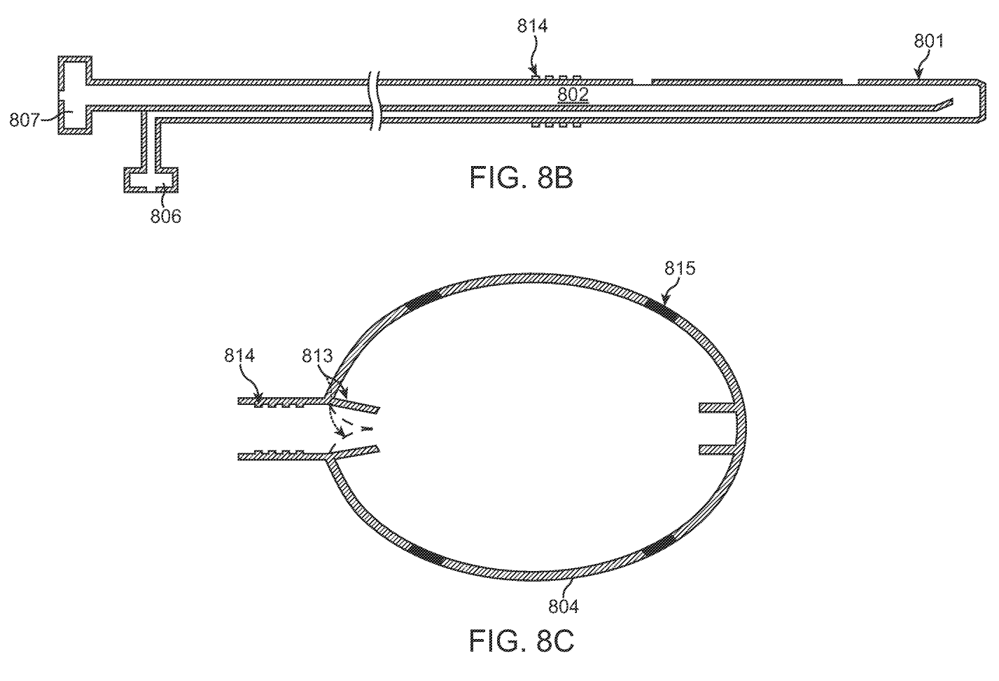

Referring to FIGS. 8A-8C, an implantable radiation delivery apparatus 800 adapted for placement at a site of tumor resection or other body cavity, comprises a detachable distal member 804 made of a non-elastic, noncompliant, bioresorbable material. Similar to the other embodiments described above, the shape of distal member 804 may be spherical, elliptical, substantially elliptical, or spiral, or comprise a plurality of small distal members to produce an overall generally spherical or elliptical shape. In a specific embodiment, the distal member 804 diameter is approximately 2-4 centimeters. However, depending on the specific requirements for a cavity to be treated, the distal member 804 could be designed larger or smaller.

The radiation delivery apparatus 800 has an elongate shaft 801 (FIG. 8B), having at least one central lumen 802 that terminates distally in one or more fluid communication ports 803. The shaft 801 is coupled to the distal member 804 (FIG. 8C) by a threaded portion 814. Filling ports 812 are provided to fill the distal member 804 with ultrasound transparent fluid. Septum 813 (shown as a duck bill structure) allows passage of the shaft 801 but prevents the fluid from escaping the inner volume defined by the distal member 804 after the shaft has been removed. The fluid communication port 803 is positioned near to the distal tip of the apparatus to ensure that all the air in the central lumen 802 is pushed forward and into fluid return channel 805. Though it is not shown in FIG. 8A, the port 803 could also be in fluid communication with the interstitium with an opening at the distal end of shaft 801. Channel 805 is formed within the walls of the apparatus, and is in fluid communication with and accessible via return fluid port 806 near the proximal end of the apparatus. Central lumen 802 is accessible via central lumen port 807 at the proximal end of the apparatus. The lumen 802 is used for first delivering the ultrasound catheter and consequently a radiation delivery device. The distal member 804 may contain radio opaque markers 815 for future diagnosis and interventions. After the final dose of radiation is delivered, the elongate shaft 801 (FIG. 8B) is decoupled from the distal member 804 and is discarded. The distal member 804 (FIG. 8C) is left behind in the treatment cavity indefinitely. Except for the radio opaque markers 815 it may contain, the distal member 804 consists of bioresorbable materials that start getting resorbed by the body within weeks of implantation.

Utility The subject invention is useful in any application where the image-guided interstitial radiation therapy is desired. One representative type of application in which the image-guided interstitial radiation therapy finds use is during and after lumpectomy procedures, standard of care today for early stage breast cancer.

Systems Also provided are systems for use in practicing the subject methods, where the systems at least include a balloon catheter device, an imaging probe, a source for providing appropriate signals to the probe to create an image, and an analyzer to collect and process the imaging data. A 3D treatment planning system along with a separate console and software may also be added as a stand alone or as an integrated part of the system to help to decide on treatment planning based on the diagnostic imaging obtained from the system.

Kits Also provided are kits for use in practicing the subject methods, where the kits typically include one or more of the above image-guidance devices, as described above. In certain embodiments, the kits at least include one imaging device. Kits may also include sheaths and source guide tubes, syringes for inflating and deflating the balloon member. Additional tubing and syringes may be necessary to remove seroma from the body cavity. Drugs and medication that can be injected into the cavity could also be included in the kit.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

It is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It should be understood that one does not have to physically “image” the cavity even if one uses ultrasound for detection and characterization of features in or around a cavity.

The information desired could be characterized by RF analysis of the ultrasound reflecting from the surrounding tissue. Similar information could be obtained by optical means, temperature measurement, X-ray, CT (computed tomography), MRI (magnetic resonance imaging), OCT (optical coherence tomography), spectrometry, biological substances and proteins, chemical analysis (such as oxygen saturation), fluorescence, to name few possibilities.

Body cavity should be understood to mean any cavity created by surgical intervention, any natural cavity, any abnormal cavity due to a birth defect or disease, and all body lumens. The term body cavity also include cavities formed by grafts and implanted devices.

Therapeutic energy delivered to the surrounding tissue does not have to be a form of “radiation” or “ionizing radiation.” All forms of therapeutic energy such as therapeutic ultrasound, heat, cryoblation, laser, and other energies are considered therapeutic energies.

What is claimed is:

1. A method for characterizing tissue surrounding a surgical resection cavity in solid tissue, said method comprising:
introducing a tissue characterization device into an interior of the surgical resection cavity; and
characterizing at least a portion of the surrounding tissue using the tissue characterization device.

2. A method as in claim 1, wherein a tumor had been surgically resected from the solid tissue to create the surgical resection cavity.

3. A method as in claim 2, wherein the solid tissue from which the tumor had been removed is breast tissue.

4. A method as in claim 1, wherein introducing the tissue characterization device comprises positioning an elongate shaft through a transcutaneous opening in tissue overlying the surgical resection cavity.

5. A method as in claim 4, wherein the tissue opening was formed in open surgery.

6. A method as in claim 4, wherein the tissue opening was formed in minimally invasive surgery.

7. A method as in claim 1, further comprising initially positioning a distal end of an introducer catheter in the surgical resection cavity, wherein the tissue characterization device is positioned through a lumen of the introducer catheter.

8. A method as in claim 7, wherein the distal end of the introducer catheter has an enlarged distal structure.

9. A method as in claim 8, further comprising expanding the enlarged distal structure so that an outer surface thereof conforms to the tissue surrounding the surgical resection cavity.

10. A method as in claim 9, further comprising contracting the enlarged distal structure prior to removing the introducer catheter from the surgical resection cavity.

11. A method as in claim 8, wherein the enlarged distal structure has a fixed geometry.

12. A method as in claim 11, wherein the enlarged distal structure is collapsed prior to removing the introducer catheter from the surgical resection cavity.

13. A method as in claim 8, further comprising drawing a vacuum in the interstitial region between the enlarged distal end and the tissue to draw the tissue against an outer surface of the enlarged distal end.

14. A method as in claim 1, wherein characterizing comprises ultrasonically scanning the tissue surrounding the surgical resection cavity.

15. A method as in claim 14, wherein the tissue characterization device comprises one or more ultrasonic transducers, wherein the transducers are rotationally and/or axially scanned within the distal end of the introduced catheter.

16. A method as in claim 1, further comprising treating at least a portion of the tissue surrounding the surgical resection cavity.

17. A method as in claim 16, wherein treatment comprises delivering ionizing or non-ionizing radiation to the tissue.

18. A method as in claim 7, further comprising positioning a treatment device through the lumen of the introducer catheter.

19. A method as in claim 18, wherein the treatment device is adapted to deliver ionizing or non-ionizing radiation.

20. A method for characterizing and treating tissue surrounding a surgical resection cavity, said method comprising:
positioning an introducer catheter transcutaneously so that a distal end is in the surgical resection cavity and a proximal end is external to the tissue;
introducing a tissue characterization device through the introducer catheter into the distal end;
characterizing at least a portion of the tissue surrounding the surgical resection cavity using the tissue characterization device;
introducing a tissue treatment device through the introducer catheter; and
treating at least a portion of the tissue surrounding the surgical resection cavity using the tissue treatment device.

21. A method as in claim 20, wherein a tumor had been surgically resected from the solid tissue to create the surgical resection cavity.

22. A method as in claim 21, wherein the solid tissue from which the tumor had been removed is breast tissue.

23. A method as in claim 20, wherein introducing the introducer catheter comprises positioning the introducer catheter through a transcutaneous opening in tissue overlying the surgical resection cavity.

24. A method as in claim 23, wherein the tissue opening was formed in open surgery.

25. A method as in claim 23, wherein the tissue opening was formed in minimally invasive surgery.

26. A method as in claim 20, wherein the distal end of the introducer catheter has an enlarged distal structure.

27. A method as in claim 26, further comprising expanding the enlarged distal structure so that an outer surface thereof conforms to the tissue surrounding the surgical resection cavity.

28. A method as in claim 27, further comprising contracting the enlarged distal structure prior to removing the introducer catheter from the surgical resection cavity.

29. A method as in claim 26, wherein the enlarged distal structure has a fixed geometry.

30. A method as in claim 29, wherein the enlarged distal structure is collapsed prior to removing the introducer catheter from the surgical resection cavity.

31. A method as in claim 26, further comprising drawing a vacuum in the interstitial region between the enlarged distal end and the tissue to draw the tissue against an outer surface of the enlarged distal end.

32. A method as in claim 20, wherein characterizing comprises ultrasonically scanning the tissue surrounding the surgical resection cavity.

33. A method as in claim 32, wherein the tissue characterization device comprises one or more ultrasonic transducers, wherein the transducers are rotationally and/or axially scanned within the distal end of the introduced catheter.

34. A method as in claim 20, wherein treatment comprises delivering ionizing or non-ionizing radiation to the tissue from the treatment device.

35. A system comprising:
an introducer catheter including a shaft having a proximal end, a distal end, a central lumen therethrough, and a distal structure positionable in a surgical resection cavity in solid tissue; and
at least one tissue characterization element insertable through the central lumen and having an active component which is positionable within the distal structure.

36. A system as in claim 35, wherein the at least one tissue characterization element is adapted to detect residual tumor cells in cavity wall tissue.

37. A system as in claim 36, wherein the tissue characterization element comprises an ultrasonic imaging transducer or array.

38. A system as in claim 35, further comprising at least one tissue treatment element adapted to treat residual tumor cells in cavity wall tissue.

39. A system as in claim 38, wherein the tissue treatment element comprises an ionizing or non-ionizing radiation source.

40. A system as in claim 35, wherein the distal structure is enlarged or enlargeable.

41. A system as in claim 40, wherein the distal structure is inflatable and deflatable.

42. A system as in claim 41, wherein the distal structure has a fixed geometry and is collapsible to a smaller diameter.

43. A system as in claim 42, wherein the distal structure is biodegradable.

44. A method for treating tissue surrounding a surgical resection cavity, said method comprising:
positioning a collapsible fixed geometry radiation delivery apparatus transcutaneously so that a distal end is in the surgical resection cavity and a proximal end is external to the tissue;
introducing a tissue treatment device through the introducer catheter; and
treating at least a portion of the tissue surrounding the surgical resection cavity using the tissue treatment device.

45. A method for treating tissue surrounding a surgical resection cavity, said method comprising:
positioning a bioresorbable radiation delivery apparatus transcutaneously so that a distal end is in the surgical resection cavity and a proximal end is external to the tissue;
introducing a tissue treatment device through the introducer catheter; and
treating at least a portion of the tissue surrounding the surgical resection cavity using the tissue treatment device.

46. An interstitial brachytherapy apparatus for delivering ionizing radiation to an internal body location comprising:
a catheter body member having a proximal end and distal end;
a volume defined by the uncollapsed state of a collapsible member
a radiation source within the apparatus generating a three-dimensional isodose profile that is substantially similar in shape to the uncollapsed shape of the collapsible member.

47. An interstitial brachytherapy apparatus for delivering ionizing radiation to an internal body location comprising:
a catheter body member having a proximal end and distal end;
a volume defined by a bioresorbable member
a radiation source within the apparatus generating a three-dimensional isodose profile that is substantially similar in shape to the bioresorbable member
